(19) United States
(12) Patent Application Publication
(10) Pub. No.: US 2018/0066888 A1
(43) Pub. Date: Mar. 8, 2018

(54) Method for Electrical Energy Storage with Co-production of Liquefied Methaneous Gas

(71) Applicant: Stanislav Sinatov, Kiryat-Ono (IL)

(72) Inventor: Stanislav Sinatov, Kiryat-Ono (IL)

(21) Appl. No.: 15/689,023

(22) Filed: Aug. 29, 2017

Related U.S. Application Data
(60) Provisional application No. 62/380,847, filed on Aug. 29, 2016.

(51) Int. Cl. F25J 1/00; C10L 3/10; C07C 9/04; F17C 7/02

(52) U.S. Cl. CPC F25J 1/0082; C10L 3/104; F17C 7/02; C07C 9/04; C10L 3/106

(57) ABSTRACT

A method for electrical energy storage with co-production of liquefied methaneous gas which comprises in combination the processes of charging the storage with liquid air through its production using an externally powered compressor train and open air auto-refrigeration cycle, storing the produced liquid air and discharging the storage through pumping, regasifying, superheating and expanding the stored air with production of on-demand power, and additionally includes a process of recovering the cold thermal energy released by regasified liquid air for controlled liquefying the methaneous gas delivered into energy storage facility at a rate and pressure consistent with those of liquid air.

### CROSS-REFERENCE TO RELATED APPLICATIONS

This application claims the benefit of U.S. Provisional Patent Application No. 62/380,847 titled “Method for Electrical Energy Storage with Co-production of Liquefied Methaneous Gas” and filed on Aug. 29, 2016.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF INVENTION

The present invention relates to the field of energy conversion technique, and more specifically to the methods enabling an improvement in the technologies intended for conversion and storage of excessive electrical energy and methaneous gas from any local source of such gaseous fuel. More particularly, the present invention relates to the methods making possible to profitably combine the operation of liquid air energy storage with co-production of liquefied methaneous gas (LMG) directly at the storage facility.

### BACKGROUND OF THE INVENTION

In modern times the electrical energy storages are becoming an integral part of the distribution grids, ensuring the on-demand and reliable supply of electricity by the intermittent renewable energy sources and providing a stable and efficient operation of the base-load fossil-fuel fired and nuclear power plants around the clock.

Amongst the known methods for energy storage able to accumulate a lot of energy and store it over a long time-period, the recently proposed methods for Liquid Air Energy Storage (LAES) (see e.c. Patent FR 2,489,411) are distinguished by a much simpler permitting process and the freedom from any geographical, land and environmental constraints, inherent in other known methods for large-scale energy storage technologies, like Pumped Hydro Electric Storage (PHES) and Compressed Air Energy Storage (CAES). In the LAES systems liquid air is produced using excessive power from the grid, stored in the small volume tanks between the off-peak and on-peak hours and re-gasified and used as effective working medium for producing a peaking power in the periods of high power demand. However, producing a liquid air during off-peak hours is an energy intensive process and many technical solutions have been proposed for an increase in air liquefaction ratio (ALR) and reducing the energy consumption and losses in this process.

One of the possible ways for improvement in LAES performance is based on the use of liquefied methaneous gas (LMG) as a medium providing capture of the cold thermal energy of the liquid air being re-gasified during LAES discharge, storage of this energy between the LAES discharge and charge and recovery of the captured cold thermal energy in the process of air liquefying during LAES charge. During these processes, the gaseous methaneous gas is delivered from the main pipeline and liquefied during LAES discharge and re-gasified and returned into the main pipeline during LAES charge. However, our investigation have revealed that amount of the LMG required to liquefy a process air during LAES charge more than twice exceeds amount of natural gas which may be liquefied during LAES discharge. In addition, a temperature of the LMG re-gasification at the enhanced pressure much exceeds a required temperature of the process air liquefying. This explains a need for use of LMG as a cold storage medium in combination with the single turbo expander-compressor based air auto-refrigeration, as described in the Patent Application No. US 2015/0226094 and applied to the LNG, or with a closed-loop refrigeration circuit using nitrogen or propane as an intermediate heat transfer fluid, as described in the UK Patent Application No. GB 2519594. The mentioned technical solutions make possible to somewhat reduce a power consumed in the process of air liquefaction. However, they practically exclude a possibility of using the produced LMG differently than for cold storage and recovery in the air liquefaction at the LAES facility.

In particular, using the produced LMG as a salable co-product of the LAES facility generated simultaneously with main LAES product (on-peak power) seems to be more justified and profitable alternative, since it makes possible not only to drastically reduce the capital costs of equipment required for methaneous gas liquefaction, but to generate both products with a moderate and acceptable energy consumption as well. Both the problems are the pressing tasks, having regard to doubling (from 600 up to 1200US$/TPA) the average first costs of the LNG plants in the past decade and unacceptably high energy consumption (400-600 kWh/ton) in the small-to-medium scale LNG production.

At the same time a proposed selling of the LMG produced requires searching for alternative means providing a desirable reduction in energy consumed in the process of air liquefying during LAES charge. For example, the use of two turbo expander-compressors which operate over different temperature levels and the different or the same pressure levels provides air auto-refrigeration at the most appropriate locations of the heat exchanger, reduces a power consumed by the compressor(s) and improves the efficiency of the air liquefaction process, as it is exemplified by the patents No. U.S. Pat. No. 4,778,497, DE 10,147,047 and US Patent Application Pub. No. 2015/0192065. Mother possibility consists in thermal assistance to the process of LAES discharge, described in the inventor's preceding provisional Patent Application No. U.S. 62/351,992 and resulting in producing an additional discharge power at the LAES facility and in corresponding increase in its round-trip efficiency. However there is a need for modernization and combination of the said means, as applied to target electric energy storage with co-production of a salable LMG product directly at the LAES facility. Finally, it is required to develop the method for controlled LMG co-production which could maximize the yield of co-product with regard to the parameters of the involved air and gas streams.

### SUMMARY OF THE INVENTION

In one or more embodiments, a proposed method for electrical energy storage with co-production of liquefied methaneous gas (LMG) may comprise in combination: a) charging the energy storage including the steps of externally powered compressing the fresh air stream up to a bottom charge pressure with its further freeing from the CO₂ and H₂O contaminants, mixing the streams of treated fresh and recirculating air streams at a bottom charge pressure thus forming a process air stream, succeeding externally powered compressing the process air up to a rated charge pressure and its final self-powered compressing up to a top charge pressure and processing between the top and bottom charge pressures in the turbo expander-compressor based open air auto-refrigeration cycle, resulting in generating a liquefied air from a part of process air at a bottom charge pressure and recirculating a rest of it for mixing with a fresh air; b) storing the produced liquid process air between the energy storage charge and discharge; c) discharging the storage including the processes of pumping the liquid air at a top discharge pressure, capturing a cold thermal energy from liquid air resulting in its re-gasifying, further thermally assisted air superheating and at least one-stage expanding down to bottom discharge pressure with on-demand producing a discharge power as a main product of the energy storage facility; d) delivering a pressurized methaneous gas from any co-located source of such gas; and e) harnessing the captured cold thermal energy of the re-gasified discharged air in the process of liquefying a delivered methaneous gas.

The invented method may differ from the known those in that a) the said self-powered compressing a process air from a rated level up to a top charge pressure may be performed through pressurizing the whole air stream air by at least two booster compressors driven by the warm and cold turbo-expanders of open air auto-refrigeration cycle and placed in tandem; b) a bulk of the delivered methaneous gas may be dried and ridded of the undesirable contaminants; c) the captured cold thermal energy of the re-gasified discharged air may be used for liquefying the treated bulk of delivered methaneous gas directly at the energy storage facility; d) the LMG may be produced at a bottom cycle pressure above atmospheric value and on-demand delivered to the customers as a saleable co-product of the energy storage facility; e) the LMG production may be controlled based on the relationship between the parameters (flow-rates and pressures) of the methaneous gas being liquefied and liquid air being re-gasified established for a given pressure of the LMG product; and f) a thermal energy assisted to energy storage facility during its discharge may be derived from combusting the untreated rest of delivered methaneous gas.

In one or more embodiments, a supplied methaneous gas may be further selected from the group consisting of but not limited to conventional natural gas, synthetic natural gas, biogas, landfill gas, tight gas, shale gas and coal bed and mine methane.

In one or more embodiments, controlling the LMG co-production directly at the energy storage facility may be further aimed at maximizing its yield up to 15%-55% of liquid discharge air flow-rate depending on the said pressures of pumped liquid discharge air, LMG produced and methaneous gas supplied.

In one or more embodiments, combusting the untreated rest of delivered methaneous gas may be performed in the integrated indirect gas fired recirculating heater placed in the closed loop, wherein circulating an intermediate heat carrier between the said heater and air superheaters at energy storage facility may be provided.

In one or more embodiments, combusting the untreated rest of delivered methaneous gas may be alternatively performed in the duct burner placed in the open circuit, wherein circulating an intermediate heat carrier from a said burner to the air superheaters at energy storage facility with a waste heat recovery of exhausted craftier may be provided.

In one or more embodiments, a bulk of the delivered methaneous gas destined for liquefaction at the energy storage facility may be dried and ridded of the undesirable contaminants at the co-located methaneous gas liquefaction plant.

In one or more embodiments, a bulk of the delivered methaneous gas destined for liquefaction at the energy storage facility may be alternatively dried and ridded of the undesirable contaminants directly at the energy storage facility.

In addition, drying and purifying a delivered methaneous gas during energy storage discharge may be performed at least partially with use of equipment employed for drying and purifying a fresh air before its liquefaction and specially designed for treating by turns both gaseous streams.

Finally, drying and purifying a delivered methaneous gas during energy storage discharge may be performed at least partially through condensing, freezing and removing the undesirable contaminants at the intermediate stage of the methaneous gas cooling and liquefaction.

### BRIEF DESCRIPTION OF THE DRAWINGS

Embodiments will hereinafter be described in detail below with reference to the accompanying drawings, wherein lie reference numerals represent like elements. The accompanying drawings have not necessarily been drawn to scale. Where applicable, some features may not be illustrated to assist in the description of underlying features.

FIG. 1 is a diagram showing schematically a possible interplay of the equipment involved into implementing the invented method for the electric energy storage (EES) with co-production of liquefied methaneous gas (LMG) directly at the EES facility.

FIG. 2 is a schematic view of the first, embodiment for implementing the EES charge with use of two turbo expander-compressor based open air auto-refrigeration cycle.

FIG. 3A is a schematic view of the second embodiment for implementing the first variant of thermally assisted EES discharge with LMG co-production directly at the energy storage facility.

FIG. 3B is a schematic view of the third embodiment for implementing the second variant of thermally assisted EES discharge with LMG co-production directly at the energy storage facility.

FIG. 4 is a diagram showing a relationship between the flow-rates of liquid air discharged and LMG produced depending on the pressures of natural gas delivered and liquid air pumped.

FIG. 5 is a diagram showing a saving in specific power consumed for LMG production at the EES facility vs. self-consumption of gas by the proposed and substituted LMG liquefaction technologies.

FIG. 6 is a diagram showing the round-trip efficiency of the EES facility with LMG co-production vs. self-consumption of gas by the proposed and substituted LMG liquefaction technologies.

### DETAILED DESCRIPTION OF THE INVENTION

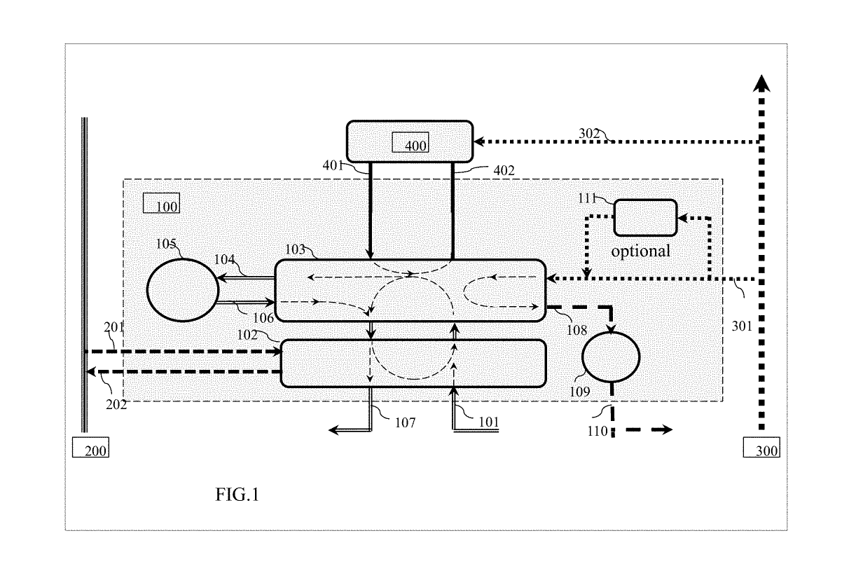

The practical realization of the proposed method for electrical energy storage (EES) with co-production of liquefied methaneous gas (LMG) may be performed through the operational interaction between all the involved facilities, equipment and utilities. FIG. 1 shows schematically a possible interplay of the facilities, equipment and utilities involved into implementing the invented method for the electrical energy storage (EES) with co-production of liquefied methaneous gas (LMG) directly at the EES facility. Here the involved facilities, equipment and utilities are designed as 100—proper EES facility, 200—electricity distribution grid, 300—distribution grid or pipeline from a local source of the methaneous gas (natural gas, synthetic natural gas, biogas, landfill gas, tight gas, shale gas and coal bed and mine methane gas) and 400—indirect gas fired recirculating heater or duct burner. A EES facility comprises the following equipment packages: 102—turbomachinery and feed air pre-treatment package, 103—heat exchangers and auto-refrigeration package, 105—liquid air storage and pumping package and 109—liquefied methaneous gas (LMG) storage. This equipment may be supplemented by the optional feed methaneous gas pre-treatment unit 111. The interaction of all mentioned elements goes on as follows.

During EES charge a fresh air is captured from atmosphere and delivered through the pipe 101 into a package 102, wherein its freeing from CO₂ and H₂O and compression up to a rated pressure are performed. Here compressing a recirculating air stream up to the said rated pressure is also conducted. A power required for air compression is consumed from the electrical grid through a line 201. A further processing of the charging air in the package 103 results in liquefying a part of process air which is delivered through a pipe 104 into a said storage 105, whereas a rest of air recirculates into a package 102.

During EES discharge a liquid air is captured from storage 105 through pipe 106 and pumped under pressure into heat exchange package 103. Here a cold thermal energy inherent in liquid air stream is captured, resulting in its re-gasification. The discharged air stream is further superheated with use of assistant thermal energy derived from combusting a gaseous methaneous fuel delivered into a said indirect gas fired recirculating heater (IGFH) or duct burner 400 from gas distribution grid 300 through a pipe 302. In the case of IGFH usage, the said assistant thermal energy is transferred from a heater 400 into a EES facility 100 by an intermediate heat carrier circulating in the closed loop 401-402 between the heater 400 and EES facility 100. Expanding a superheated air stream in the turbomachinery of package 102 leads to generation of power delivered into grid through a line 202. The expanded air is exhausted into atmosphere through a pipe 107.

Of total amount of gaseous fuel delivered into EES facility through pipes 301 and 302, only ˜3-5% is directed through pipe 302 to the IGFH or duct burner 400, whereas the most (˜95-97%) of delivered gas is directed through a pipe 301 for its conversion into a saleable liquefied methaneous gas (LMG) co-product. If needed, a gas destined for liquefaction may be treated of CO₂, H₂O and other impurities in the optional gas pre-treatment unit 111. Alternatively already treated gas may be delivered from the co-located LNG plant or biogas treatment unit. A cold thermal energy required for methaneous gas liquefaction is extracted in the package 103 from a liquid air stream being re-gasified during FPS discharge. The generated LMG is directed through a pipe 108 into the storage tank 109 and on-demand delivered through a pipe 110 to the customers as a saleable co-product of the EES facility. Maximizing the LMG co-production rate is achieved through controlling a relationship between the flow-rates of methaneous gas, which is delivered through a pipe 301 and destined for liquefaction, and liquid air, which is delivered through a pipe 106 and destined for re-gasification. The said relationship may be selected in the range from 0.15 to 0.55, depending on the pressures of pumped liquid discharged air, LMG produced and methaneous gas supplied.

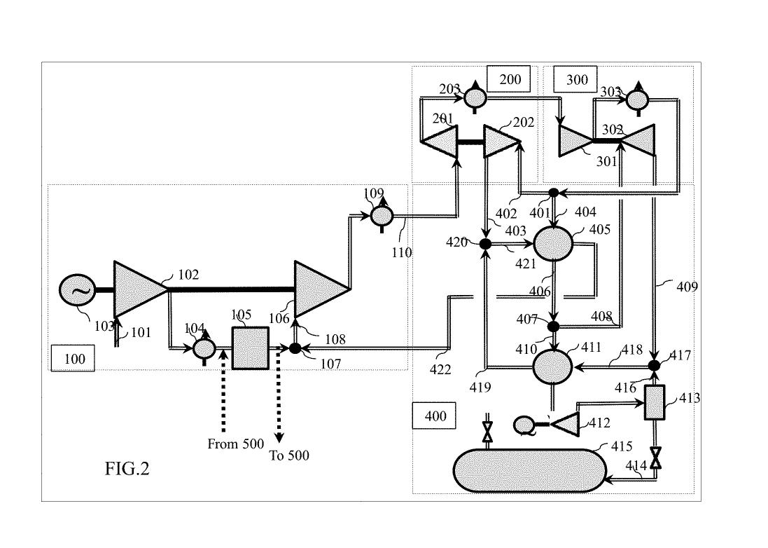

FIG. 2 is a schematic view of the first embodiment for implementing the EES charge with use of two turbo expander-compressor based open air auto-refrigeration cycle. Here, sequential further compressing the whole of pre-pressurized process air stream from a rated level up to a top charge pressure is performed in two booster compressors placed in-series and driven by the warm and cold turbo-expanders of open air auto-refrigeration cycle. This approach makes possible to provide energy consumption during EES charge at the level comparable to that provided by the single turbo expander-compressor based open air auto-refrigeration cycle, which however uses an assistant LNG cold potential. The charge of EES facility may be performed in this case with use of the following equipment packages:
100—compressor train with associated equipment
200—warm turbo expander-booster compressor train
300—cold turbo expander-booster compressor hair
400—liquefaction, separation and storage equipment package.

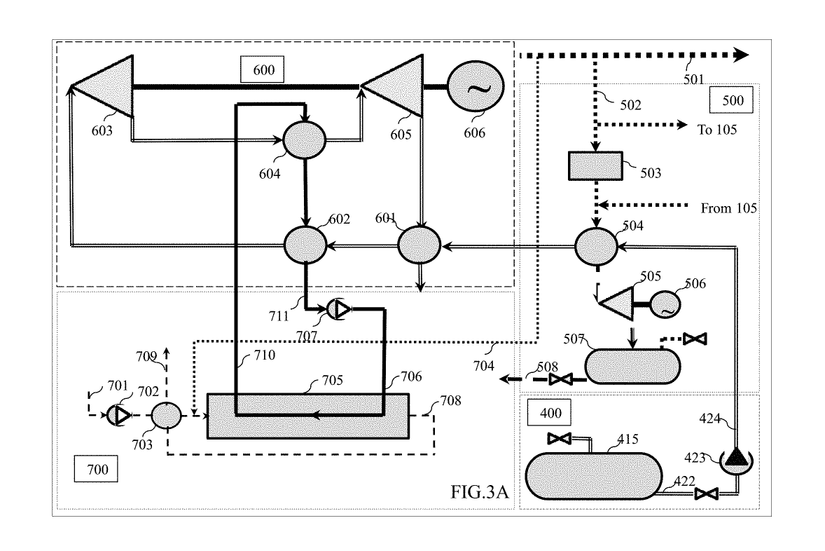

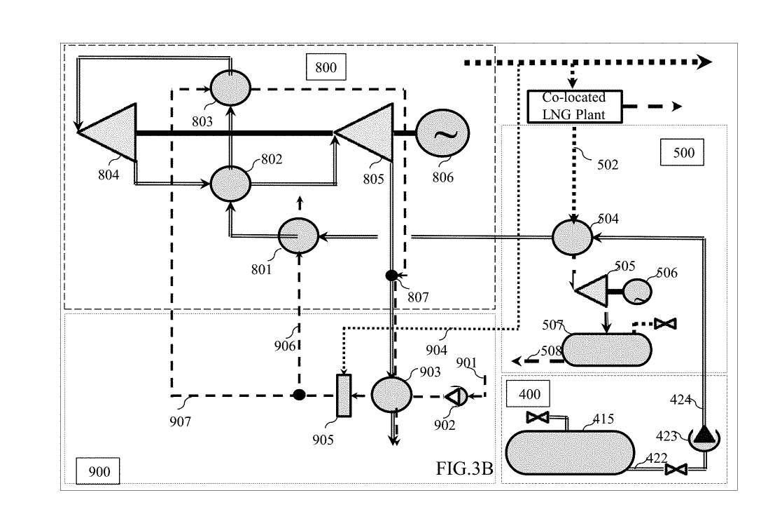

According to the present invention, compressor train is designed as two-stage turbomachinery, wherein the first compression stage 102 and second compression stage 106 are driven by the common electric motor 103. A fresh air from atmosphere is delivered through a pipe 101 into the first compression stage 102 and pressurized up to a bottom charge pressure. Train is equipped with intercooler 104 and inter-cleaner (adsorber) 105 for capture of moisture and carbon dioxide from a pressurized fresh air. The adsorber may be used also for at least partial treatment of methaneous gas delivered from the package 500 (see FIG. 3A) during EES discharge. At the outlet of adsorber (point 107) the cooled and cleaned fresh air is mixed with a recirculating air stream 422 delivered under a bottom charge pressure from a package 400, so forming a process air stream 108, which is further compressed in the second compression stage 106 up to a rated pressure level. A removal of compression heat in the intercooler 104 and aftercooler 109 is performed by an ambient air or water. If needed, the second compression stage 106 may be designed in the intercooled configuration.

Further compressing the entire process air stream up to a top charge pressure is sequentially performed in the booster compressors 201 and 301 driven by the warm and cold turbo-expanders 202 and 302 with cooling the air after each compressor in the heat exchangers 203 and 303 accordingly. At the said top charge pressure the process air stream is directed to the point 401, wherein it is divided into two streams 402 and 404. The first extracted part of process air (stream 402) is expanding down to a bottom charge pressure in the said warm turbo-expander 202 with an accompanied deep cooling of expanded air stream 403. The rest of process air (stream 404) is delivered into a deep cooler 405, wherein its temperature decreased substantially below 0° C. with a recirculating air stream. At the outlet of deep cooler 405 (point 407) the deeply cooled rest of process air stream 406 is further divided into two streams (408 and 410). The second extracted part of process air (stream 408) is expanding in the said cold turbo expander 302 down to a bottom charge pressure with an accompanied deep cooling of expanded air stream 409 down to a temperature below a temperature of the stream 403. The definitive rest 410 of process air is additionally cooled and fully liquefied in the air liquefier 411. The liquefied definitive rest of process air is further directed into a generator-loaded turbine 412, wherein it is expanded down to a bottom charge pressure with an accompanied final cooling of expanded air down to bottom charge temperature. A bottom charge pressure is selected at a level exceeding atmospheric pressure by 1-7 bar. An air separator 413 installed at the outlet of expander 412 is used to separate the liquid and gas phases of the finally expanded and cooled definitive rest of process air. The liquid air stream 414 is directed to the pressurized liquid air vessels 415, wherein it is stored at the bottom charge pressure and temperature between the EES charge and discharge. The gaseous air stream 416 is directed to the point 417, wherein its mixing with an expanded and cooled second extracted part 409 of process air is performed. This results in formation of an initial part 418 of recirculating air stream at a bottom charge pressure. The said initial part 418 of recirculating air stream is further used for the final cooling and liquefying the definitive rest 410 of process air in the air liquefier 411, causing the accompanied heating the outgoing stream 419. The said stream 419 is mixed at the point 420 with the first extracted part 403 of process air coming from the warm expander 202, resulting in final formation of the recirculating stream 421. The said recirculating stream 421 is further used for said cooling the rest 404 of process air in the deep cooler 405, causing the accompanied further heating the outgoing stream 422. The recirculating air stream 422 outgoing from the deep cooler 405 at a bottom charge pressure is directed into the package 100 for mixing with a fresh air stream at the point 107.

FIG. 3A is a schematic view of the second embodiment for implementing the first variant of thermally assisted EES discharge with LMG co-production directly at the energy storage facility according to the present invention. The approach to a thermally assisted EES discharge being proposed in the present invention is basically the same as that described in the inventor's preceding provisional Patent Application No. U.S. 62/442,457. But contrary to the mentioned patent application, the sole source of assistant thermal energy in the present technical solution is an integrated indirect gas fired recirculating heater and emphasis is on the minimization of amount of the fuel consumed by heater for generation of assistant thermal energy. The EES in the FIG. 3A is exemplified by a facility, which includes the following equipment packages:

Here the following equipment packages are used:
400—air liquefaction, separation and storage equipment
500—methaneous gas liquefaction and LMG storage equipment
600—air expander train
700—indirect gas fired recirculating heater package.

Operation of the EES facility in discharge operation mode is performed as follows. A stream 422 of liquid process air is extracted at a bottom discharge pressure from the storage 415 and pumped by a pump 423 up to top discharge pressure selected in the range between 10 and 200 bar. The discharged air stream 424 is delivered into a package 500 which is destined for liquefaction of any pressurized methaneous gas supplied from a local source 501 of such gas. In the FIG. 3A this local gas source is exemplified by a natural gas distribution grid 501. The feed gas destined for liquefaction at the EES facility is delivered through a pipe 502 into pre-treatment unit 503, wherein it is dryed and purified. Alternatively, the pre-treatment of methaneous gas during EES discharge may be at least partially performed in the adsorber 105 (see FIG. 2), which is used for pre-treatment of fresh air during EES charge. A pressure of supplied gas may be varied between 10 and 150 barA and should be consistent with the said top discharge pressure of process air to maximize a yield of LMG produced. At the LMG pressure selected in the range between 2 and 12 barA a flow-rate of the LMG produced may reach 15-55% of a flow-rate of discharged air flow-rate. As this takes place, a LMG production yield may be increased either through a decrease in selected top discharged air pressure at a given pressure of supplied methaneous gas, or through an increase in pressure of supplied methaneous gas at a given top discharged air pressure. The latter approach may be realized through using an additional gas compressor in the pre-treatment unit 503, whereas for realization of the first-named approach the appropriate adjustment of the liquid air pump 423 and selection of a proper expander train 600 configuration are required.

To provide the LMG production directly at the EES facility a pumped discharged air stream 424 is delivered into a methaneous gas liquefier—discharged air regasifier 504. Exchange of thermal energy between the discharged liquid air and supplied methaneous gas in this heat exchanger results in liquefaction of entire methaneous gas stream 502 and regasification of entire discharged air stream 424. The latter is directed from the regasifier 504 to the air expander train 600, wherein it first preheated in the recuperator 601, and thereafter superheated in the heat exchanger 602. The following expanding the superheated air stream is performed in the at least one-stage expander train 600 whose configuration is selected with regard to a top discharged air pressure provided by a pump 423. At a said pressure exceeding 40-45 barA a two-stage expander train configuration with reheating a discharged air between the stages above 600° C. is preferable. Such a configuration makes possible to use a modernized back-pressure steam turbine as the first high-pressure stage of air expander. At the same time it requires that a temperature of discharged air at the inlet of first stage do not exceeding 600° C.

As shown in the FIG. 3A, the superheated air is expanded in the first stage 603 of expander train and reheated in the heat exchanger 604 at a sacrifice of heat exchange with the intermediate heat carrier circulating in the closed loop between the expander train 600 and indirect gas fired heater package 700. A standard widely-known design of the indirect gas fired recirculating heater is used in the present invention. The air required for combustion of fuel is captured from atmosphere through a pipe 701, slightly pressurized in a blower 702, preheated in the recuperator 703 and mixed with a gaseous fuel delivered through a pipe 704. A heat released during combustion of fuel in a proper heater 705 is used to increase a temperature of an intermediate heat carrier delivered into a heater through a pipe 706 and outgoing through a pipe 710. Recirculating a stream of the combustion products 708 and recovering its waste heat in recuperator 703 provides a high efficiency of assistant thermal energy production. A stream of intermediate heat carrier escaping the reheater 604 is directed to the superheater 602 and further through a blower 707 and pipe 706 to the heater 705. In its turn, the reheated air stream is expanded down to a bottom discharge pressure near atmospheric with an accompanied its cooling at the outlet of stage 605. A work performed by the expanded air stream in the stages 603 and 605 is converted into electric power by the shaft-coupled generator 606. A process air escaping the second stage 605 of expander train possesses a sufficient thermal energy to be used in the recuperator 601 for the said preheating a discharged air upstream of the superheater 602.

A liquefied methaneous gas is directed from the heat exchanger 504 to the generator 506 loaded liquid gas expander 505, wherein its depressurization down to a selected storage pressure with a final cooling are performed. This results in forming the LMG co-product which is stored in the pressurized storage tank 507 and on-demand delivered to the customers through a pipe 508.

FIG. 3B is a schematic view of the third embodiment for implementing the second variant of thermally assisted EES discharge with LMG co-production directly at the energy storage facility according to the present invention. The approach to a thermally assisted EES discharge being proposed in the present invention is basically the same as that described in the inventor's preceding provisional Patent Application No. US U.S. 62/442,457. But contrary to the mentioned patent application, the sole source of assistant thermal energy in the present technical solution is an integrated direct gas fired recirculating heater and emphasis is on the minimization of amount of the fuel consumed by its burner for generation of assistant thermal energy. The EES in the FIG. 3B is exemplified by a facility, which includes the following equipment packages:
400—air liquefaction, separation and storage equipment
500—methaneous gas liquefaction and LMG storage equipment
800—air expander train
900—direct gas fired recirculating heater package.

Operation of the EES facility in discharge operation mode is performed as follows. A stream 422 of liquid process air is extracted at a bottom discharge pressure from the storage 415 and pumped by a pump 423 up to top discharge pressure selected in the range between 10 and 200 bar. The discharged air stream 424 is delivered into a package 500 which is destined for liquefaction of any pressurized methaneous gas supplied from a local source of such gas. In the FIG. 3B this local gas source is exemplified by a co-located LNG plant which is not a part of the EES facility. This plant is equipped with the equipment performing the drying and purification of entire stream of feed gas delivered from the distribution grid. One part of treated gas is liquefied at the LNG plant, whereas another part is delivered through a pipe 502 for liquefaction at the EES facility. To maximize a yield of LMG produced, a pressure of treated gas delivered from the LNG plant should be consistent with the top discharge pressure of process air at the EES facility, as described above.

To provide the LMG production directly at the EES facility a pumped discharged air stream 424 is delivered into a methaneous gas liquefier—discharged air regasifier 504. Exchange of thermal energy between the discharged liquid air and supplied methaneous gas in this heat exchanger results in liquefaction of entire methaneous gas steam 502 and regasification of entire discharged air stream 424. The latter is directed from the regasifier 504 to the two-stage air expander train 800, wherein it is first superheated in the heat exchanger 801 up to top discharge temperature and thereafter is used for reheating the discharged air stream in the heat exchanger 802 between the expander stages 804 and 806. After following repeated superheating in the heat exchanger 803 up to intermediate discharge temperature this air is expanded firstly in the high pressure stage 804 of expander train, reheated in the said heat exchanger 802 up to a temperature close to top discharge value and then expanded in the low pressure stage 805 of expander train.

As shown in the FIG. 3B, superheating the discharged air in the heat exchangers 801 and 803 is performed a sacrifice of heat exchange with the intermediate heat carrier circulating in the open loop between the expander train 800 and direct gas fired recirculating heater package 900. A standard widely-known design of industrial heater is used in the present invention. The air required for combustion of fuel is captured from atmosphere through a pipe 901, slightly pressurized in a blower 902, preheated in the recuperator 903 and mixed with a gaseous fuel delivered through a pipe 904. A heat released during combustion of fuel in a proper heater 905 is used to increase a temperature of an intermediate heat carrier delivered into a heater from recuperator 903. A greater part 906 of the combustion products is directed to the heat exchanger 801 and exhausted from it after deep cooling. A lesser part 907 of the combustion products is directed to the heat exchanger 803 and leaves it at a reasonable high temperature. The discharged air stream escapes the low-pressure stage 805 of expander train also at the enhanced temperature. Combining these two streams at the point 807 and recovery of their waste heat in the recuperator 903 provides a high thermal efficiency of the direct fired heater 905 operation.

A liquefied methaneous gas is directed from the heat exchanger 504 to the generator 506 loaded liquid gas expander 505, wherein its depressurization down to a selected storage pressure with a final cooling are performed. This results in forming the LMG co-product which is stored in the pressurized storage tank 507 and on-demand delivered to the customers through a pipe 508.

### INDUSTRIAL APPLICABILITY

The performances of Electrical Energy Storage (EES) facility with co-production of liquefied methaneous gas (LMG) directly at the facility (see FIG. 1) are presented below. The calculation of these performances has been performed as applied to 12 hr of EES charge duration and 12 hr of its discharge with the possible generating 7-9 MW of peaking power and ˜14-15 t/h of the LMG co-product, equivalent to annual LMG capacity of 0.055-0.060 MTPA. Such LMG yield is inherent for the small-scale liquefaction plants, distinctive in the enhanced specific installed costs and impressive specific power consumption. Therefore the proposed method for LMG co-production directly at the EES facility, drastically reducing both the indexes, may be especially attractive for the small-scale LMG plant designers and operators. At the same time this method may be successfully used also in construction and operation of the large-scale EEC facilities, generating the on-demand power and LMG co-product at the rates of 400-500 MW and 2-3 MTPA respectively.

In the conducted feasibility study it was assumed that the EES facility is supplied with pressurized and treated natural gas from a co-located pre-treatment unit or a part of pre-treated gas is delivered from the adjacent LNG plant. A required amount of untreated gas is delivered to the duct burner placed into open circuit of intermediate heat carrier with recuperator of exhaust waste heat (see FIG. 3B) The facility itself is equipped with the equipment, providing the EES charge with use of two turbo expander-compressor based open air auto-refrigeration cycle (see FIG. 2) and EES discharge with use of thermally assisted 2-stage expander train (see FIG. 3B).

The given and assumed technical data used in numerical simulation of the EES facility performance are listed in the Table 1 below.

TABLE 1

| Parameter | Unit | Data |
|---|---|---|
| EES facility discharge power at the identical number of | MWe | 7-9 |
| charge and discharge hours | | |
| Total compressor polytropic & mechanical efficiency | % | 87 |
| Total expander adiabatic & mechanical efficiency | % | 87 |
| Total coupling & electric motor efficiency of | % | 97.5 |
| turbomachinery | | |
| Isentropic liquid air and gas expander efficiencies | % | 85 |
| Isentropic liquid air pump efficiency | % | 80 |
| Small generator/motor electric efficiency | % | 96 |
| Compressor train outlet pressure | barA | 34.8 |
| Top charge pressure | barA | 61.7 |
| Bottom charge pressure | barA | 6.7 |
| Top discharge temperature | ° C. | 870 |
| Top discharge pressure | barA | 150 |
| Assumed pressure drop in piping | barA | 0 |
| Assumed pressure drop in each heat exchanger | barA | 0.025 |
| Discharged air temperature at HP expander inlet | ° C. | 565 |
| Discharged air pressure at HP expander outlet | barA | 43 |
| LNG co-product pressure | barA | 7 |
| Natural gas inlet pressure | barA | 66 |

In their turn, the calculated performance resulted from numerical simulation of the EES facility charge are presented in the Table 2. Here the following designations are used for the charge process:

G_{PA} and G_{LA}—flow-rates of process air and liquid air produced, kg/s;
W_{FAC}, W_{MAC} and W_{LAE}—mechanical power consumed by the feed and main air compressors and produced by the liquid air expander, kWm;
W_{CH}—total electric power consumed during EES charge, kWe;
ALR=G_{LA}/G_{PA}—air liquefaction ratio, %; and
ω_{CH}=W_{GH}/(G_{LA}×3.6)—specific power consumed by the EES facility during its charge, kWh/ton of liquid air.

TABLE 2

| G_{PA} | G_{LA} | W_{FAC} | W_{MAC} | W_{LAE} | W_{CH} | ω_{CH} | ALR | Remarks |
|---|---|---|---|---|---|---|---|---|
| 48 | 8.9 | 2,283 | 10,631 | 53 | 13,194 | 412 | 18.5 | Uncooled |
| | | | | | | | | AC |

The calculated performance of the discharge process are presented in the Table 3, wherein the following designations are used:

G_{NG} and G_{LNG}—flow-rates of natural gas delivered and liquefied natural gas produced, kg/s;
W_{HPAE}, W_{LPAE} and W_{LGE}—mechanical power produced by the high and low pressure air expanders and liquid gas expander, kWm;
W_{AUX}—mechanical power consumed by the liquid air pump and air blower, kWm;
W_{DCH}—electric power produced during EES discharge, kWe;
GLR=G_{LNG}/G_{NG}—gas liquefaction ratio, %; and
ω_{DCH}=W_{DCH}/(G_{LA}×3.6)—specific discharge produced by the EES facility during its discharge, kWh/ton of liquid air.

TABLE 3

| G_{NG} | G_{LNG} | GLR | W_{HPAE} | W_{LPAE} | W_{LGE} | W_{AUX} | W_{DCH} | ω_{DCH} |
|---|---|---|---|---|---|---|---|---|
| 4.04 | 4.04 | 100 | 2,048 | 6,102 | 48 | 297 | 7,683 | 240 |

As may be seen from the data presented in the Tables 1-3, the amount of LNG produced directly at the EES facility during its discharge constitutes ˜4 kg/s or 45.4% of an amount of liquid air produced during EES facility charge for the given conditions mentioned above.

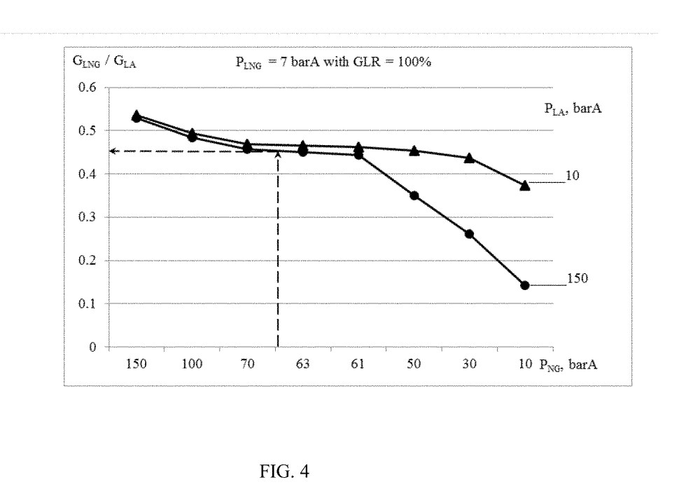

The achievable relationships between the amounts of LNG and liquid air produced for a wide range of the liquid air and feed NG pressures at the fixed pressure of LNG produced are presented in the FIG. 4.

The concluding analysis of the calculated EES facility performance is presented below, wherein the following designations are used:

G_{SC}—amount of fuel combusted in duct burner and supposedly used for LNG production only, kg/s;
LHV=47,141 kJ/kg—assumed low heating value of fuel used;
Qth=G_{SC}×LHV—heat input in the EES facility, kWth;
G_{SC}/(G_{SC}−G_{LNG})×100—relative amount of fuel self-consumed at the EES facility and substituted LNG plants, %;
η_{EL}=33%—assumed fuel-to-power conversion efficiency at the EES facility and substituted LNG plants;
W_{LNG}=G_{SC}×LHV×η_{EL}—power consumed for gas liquefaction at the EES facility and substituted LNG plants, kWe;
ω_{LNG}=W_{LNG}/(G_{LNG}×3.6)—specific power consumed in liquefaction process, kWh/t of LNG;
ΔW_{(LNG)i}=W_{(LNG)1}−W_{(LNG)3}, where i=2, 3, 4—saving in power consumption at the substituting EES facilities, kWe;
ΣW_{(DCH)i}=W_{DCH}+ΔW_{(LNG)i}—total discharge power of substituting EES facilities, kWe;
RTE_{REC}=(ΣW_{DCH}/W_{CH})×100—recasted round-trip efficiency of substituting EES facilities, %; and
RTE_{GRID}=W_{DCH}/W_{CH}—grid round-trip efficiency of basic EES facility.

A thermal assistance to operation of the EES facility in discharge mode is performed through combustion of only 4% of total amount of natural gas delivered to the EES facility. This is far less than is self-consumed at the known, especially small-scale, LNG plants, wherein natural gas self-consumption ranges from 7% to 11% and above depending on a type of used liquefaction technology and size of LNG plant. That much energy saving, provided by the LNG liquefaction directly at the EES facility, should be reflected in the calculation of its recasted round-trip efficiency.

For these purposes a fuel self-consumed both at the basic EES facility and at the substituted LNG plants should be converted into power consumed for gas liquefaction. At the medium-to-large scale LNG plants this may be effected using the integrated simple cycle gas turbine plants with fuel-to-power conversion efficiency between 34% and 36%. The small-to-medium scale LNG plants consume often a power for their operation from the grid, wherein average fuel-to-power conversion efficiency is about 31%. In the considered case a value of such efficiency is assumed to be equal to 33%. Division of the calculated values of consumed power by the amount of LNG produced makes possible to determine a specific power consumed in liquefaction process. In addition, through comparison of the calculated values of consumed power for each known liquefaction technology with a power arbitrarily consumed for gas liquefaction at the basic EES facility, a possible saving in power consumption may be determined. This saved power should be added to the EES discharge power determined in the Table 3, after which a total discharge power has to be divided by the charge power determined in the Table 2. It makes possible to define a recasted round-trip efficiency of the substituting EES facilities with LNG co-production.

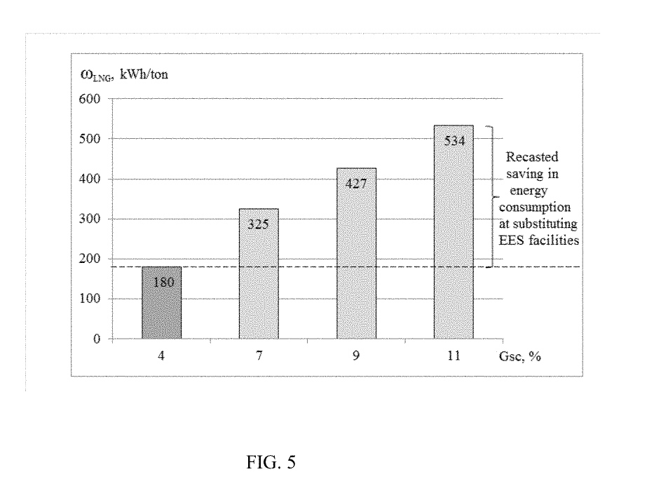

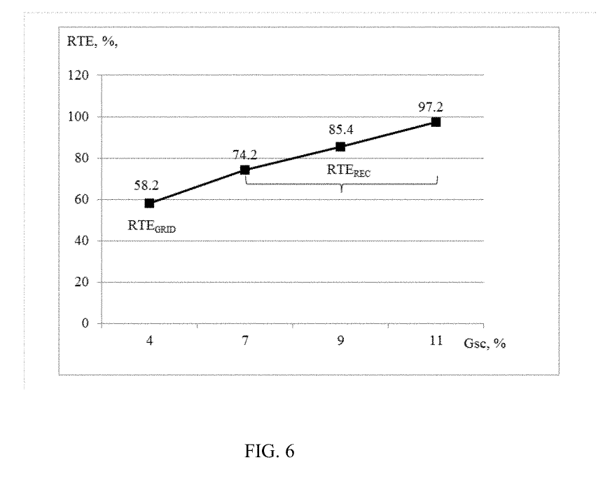

The results of calculation are presented in the Table 4 and FIG. 5-6.

TABLE 4

| | | | |
|---|---|---|---|
| | | Basic EES | Possible data of the substituted |
| Parameters | Unit | facility | LNG plants |

| i | | 1 | 2 | 3 | 4 |
|---|---|---|---|---|---|
| G_{LNG} | kg/s | 4.04 | 4.04 | 4.04 | 4.04 |
| Fuel self-consumption | % | 4 | 7 | 9 | 11 |
| G_{SC} | kg/s | 0.168 | 0.304 | 0.399 | 0.498 |
| Qth | kWth | 7,914 | 14,307 | 18,786 | 23,490 |
| W_{LNG} | kWe | 2,612 | 4,721 | 6,199 | 7,752 |
| ω_{LNG} | kWh/ton | 180 | 325 | 427 | 534 |
| | | Basic EES | Possible data of the substituting | | |
| | | facility | EES facilities | | |
| ΔW_{LNG} | kWe | 0 | 2,110 | 3,588 | 5,140 |
| W_{DCH} | kWe | 7,682 | 7,682 | 7,682 | 7,682 |
| ΣW_{DCH} | kWe | 7,682 | 9,792 | 11,270 | 12,822 |
| RTE_{REC} | % | | 74.2 | 85.4 | 97.2 |
| RTE_{GRID} | % | 58.2 | | | |

As evident from the FIG. 5, a proposed co-production of the LNG directly at the EES facility instead of commonly-used its production at the LNG plants provides reduction of specific power consumption from 325-534 kWh/t to 180 kWh/t, that is approximately 2-3 times. In addition, CAPEX of the LNG co-production at the EES facility could be not more than 50% of the CAPEX of LNG plant, since there is not a need for the expensive liquefaction and refrigeration equipment. Finally, the mentioned saving in power required for LNG co-production during EES discharge implies practically a retention of highly valuable peaking power in the grid. This effect may be estimated through a corresponding increase in the discharge power of the substituting EES facilities and resulting enhancement of their recasted round-trip efficiency. As this takes place, the less is an efficiency of the substituted LNG plant, the more is recasted efficiency of the substituting EES facility. Reference to FIG. 6 shows, that if the EES facility substitutes LNG plants, having self-consumption of fuel from 7% to 11%, the round-trip efficiency of such substituting EES facility may be in the range from 74.2% to 97.2%, significantly exceeding the grid round-trip efficiency of the basic EES facility which is equal to 58.2%.

It should be noted that the term “comprising” does not exclude other elements or steps and “a” or “an” do not exclude a plurality. It should also be noted that reference signs in the claims should not apparent to one of skill in the art that many changes and modifications can be effected to the above embodiments while remaining within the spirit and scope of the present invention.

What is claimed as new is:

1. A method for electrical energy storage with co-production of liquefied methaneous gas (LMG), comprising in combination:
charging the energy storage including the steps of externally powered compressing the fresh air stream up to a bottom charge pressure with its further freeing from the CO₂ and H₂O contaminants, mixing the streams of treated fresh and recirculating air streams at a bottom charge pressure thus forming a process air stream, succeeding externally powered compressing the process air up to a rated charge pressure and its final self-powered compressing up to a top charge pressure and processing between the top and bottom charge pressures in the turbo expander-compressor based open air auto-refrigeration cycle, resulting in generating a liquefied air from a part of process air at a bottom charge pressure and recirculating a rest of it for mixing with a fresh air;
storing the produced liquid air between the energy storage charge and discharge;
discharging the storage including the processes of pumping the liquid air at a top discharge pressure, capturing a cold thermal energy from liquid air resulting in its re-gasifying, further thermally assisted air superheating and its at least one-stage expanding down to bottom discharge pressure with on-demand producing a discharge power as a main product of the energy storage facility;
delivering a pressurized methaneous gas from any available source of such gas;
harnessing the captured cold thermal energy of the re-gasified discharged air in the process of liquefying a delivered methaneous gas; and
wherein the improvement comprises in combination:
the said self-powered compressing a process air from a rated level up to a top charge pressure is performed through pressurizing the whole air stream air by at least two booster compressors driven by the warm and cold turbo-expanders of open air auto-refrigeration cycle and placed in tandem;
a bulk of the delivered methaneous gas is dried and ridded of the undesirable contaminants;
the captured cold thermal energy of the re-gasified discharged air is used for liquefying the treated bulk of delivered methaneous gas directly at the energy storage facility;
the LMG is produced at a bottom cycle pressure above atmospheric value and on-demand delivered to the customers as a saleable co-product of the energy storage facility;
the LMG production is controlled based on the relationship between the parameters (flow-rates and pressures) of the methaneous gas being liquefied and liquid air being re-gasified established for a given pressure of the LMG product; and
a thermal energy assisted to energy storage facility during its discharge is derived from combusting the untreated rest of delivered methaneous gas.

2. A method as in claim 1, wherein a delivered methaneous gas is selected from the group consisting of but not limited to conventional natural gas, synthetic natural gas, biogas, landfill gas, tight gas, shale gas and coal bed and mine methane.

3. A method as in claim 1, wherein controlling the LMG co-production directly at the energy storage facility is aimed at maximizing its yield up to 15%-55% of liquid discharge air flow-rate depending on the said pressures of pumped liquid discharge air, LMG produced and methaneous gas supplied.

4. A method as in claim 1, wherein combusting the untreated rest of delivered methaneous gas is performed in the integrated indirect gas fired recirculating heater placed in the closed loop, wherein circulating an intermediate heat carrier between the said heater and air superheaters at energy storage facility is provided.

5. A method as in claim 1, wherein combusting the untreated rest of delivered methaneous gas is performed in the integrated direct gas fired recirculating heater placed in the open circuit, wherein circulating an intermediate heat carrier from a said heater to the air superheaters at energy storage facility with a waste heat recovery of exhausted carrier are provided.

6. A method as in claim 1, wherein a methaneous gas destined for liquefaction at the energy storage facility is dried and ridded of the undesirable contaminants at the co-located liquefaction plant.

7. A method as in claim 1, wherein a methaneous gas destined for liquefaction at the energy storage facility is dried and ridded of the undesirable contaminants directly at the energy storage facility.

8. A method as in claims 1 and 7, wherein drying and purifying a delivered methaneous gas during energy storage discharge is performed at least partially with use of equipment employed for drying and purifying a fresh air before its liquefaction and specially designed for treating by turns both gaseous streams.

9. A method as in claims 1 and 7, wherein drying and purifying a delivered methaneous gas during energy storage discharge is performed at least partially through condensing, freezing and removing the undesirable contaminants at the intermediate stage of the methaneous gas cooling and liquefaction.
